(19) United States
(12) Patent Application Publication
(10) Pub. No.: US 2007/0026383 A1
(43) Pub. Date: Feb. 1, 2007

(54) Methods and compositions for extracting membrane proteins

(75) Inventors: Vladimir Trubetskoy, Middleton, WI (US); Olga Trubetskoy, Middleton, WI (US)

Correspondence Address: Medlen & Carroll, LLP, Suite 350, 101 Howard Street, San Francisco, CA, 94105, US

(73) Assignee: Quintessence Biosciences, Inc., Madison, WI (US)

(21) Appl. No.: 11/487,833

(22) Filed: Jul. 17, 2006

Related U.S. Application Data
(60) Provisional application No. 60/699,947, filed on Jul. 15, 2005.

(51) Int. Cl. C12Q 1/00; G01N 33/53

(52) U.S. Cl. 435/4; 435/7.1

(57) ABSTRACT

The present invention provides methods and compositions for the isolation and use of membrane proteins and other membrane associated molecules (e.g., peptides, carbohydrates, lipids, or combinations thereof). In particular, the present invention provides amphiphilic polymer compositions and methods of using the compositions to solubilize, enrich and isolate components of biological membranes, including membrane proteins, while retaining function and/or activity of the component.

The present invention claims priority to U.S. Provisional Application Ser. No. 60/699,947, filed Jul. 15, 2005, herein incorporated by reference in its entirety.

### FIELD OF THE INVENTION

The present invention provides methods and compositions for the isolation and use of membrane proteins and other membrane associated molecules (e.g., peptides, carbohydrates, lipids, or combinations thereof). In particular, the present invention provides amphiphilic polymer compositions and methods of using the compositions to solubilize, enrich and isolate components of biological membranes, including membrane proteins, while retaining function and/or activity of the component.

### BACKGROUND OF THE INVENTION

The different classes of membrane proteins serve many essential functions and include G-protein coupled receptors (GPCRs), transporters, ion channels, and cell surface recognition proteins. Many enzymes are also membrane-bound, including some kinases and drug-metabolizing enzymes (DMEs) such as the cytochrome P450s and UDP-glycosyltransferases (UGTs). The importance of membrane proteins is illustrated by the fact that up to 70% of known drug targets are membrane proteins and 30% of the human genome encodes membrane proteins.

Membrane proteins include drug metabolizing enzymes, intracellular signaling proteins such as G proteins, as well as additional receptor proteins. Membrane proteins are involved in many important biological functions. The study of membrane proteins is hindered by their low solubility and aggregation in aqueous solutions commonly used to purify proteins. Currently available methods for solubilization often result in alteration of protein structure, as well as substantial reduction or elimination of protein function and/or activity.

For example, several classes of membrane proteins and enzymes are involved in the uptake, metabolism, and clearance of drugs and other therapeutic substances through Phase I oxidative metabolism by cytochrome P450s (P450s), Phase II conjugative metabolism by UDP-glycosyltransferases (UGTs), and Phase III transport across the cell membrane by drug transporters. These membrane proteins are especially important to the pharmaceutical industry during lead optimization and are used to identify compounds with optimal pharmacokinetics and toxicology profiling, and to select the best drug candidates for preclinical studies. In vitro screening procedures utilizing these membrane proteins aim to reduce side effects due to adverse drug reactions (ADRs) by eliminating problematic compounds or predicting potentially toxic drug-drug interactions. However, research involving membrane proteins faces serious obstacles, including protein instability, significant spectrophotometric light scattering, low signal to noise ratios in fluorescent assays, and high variability in assay methods. The instability of membrane proteins, especially detergent-solubilized membrane proteins, is a major problem in membrane biology and in developing assays such as drug screening assays for membrane targets.

Glucuronidation is an important detoxification pathway that can affect pharmacokinetic and pharmaco-dynamic properties of therapeutic agents and produce reactive metabolites and potential drug-drug interactions. No suitable robust assays for assessing drug glucurondations and related drug-drug interactions exist. The need to study the complexity of UGT-related adverse drug reactions and toxicity in all population groups, including the young, the elderly, and carriers of polymorphic variants demands development of efficient assays (e.g., high throughput assays) for small molecule glucuronidation. Attempts to develop robust UGT assays have failed, due to the challenges of working with dilute membrane enzyme preparations, including low enzyme stability, significant light scattering, low signal to noise ratios, and low assay variability.

Therefore, improved methods for isolating and stabilizing membrane proteins for use in drug screening and other applications are needed.

### SUMMARY OF THE INVENTION

The present invention provides methods and compositions for the isolation and use of membrane proteins and other membrane associated molecules (e.g., peptides, carbohydrates, lipids, or combinations thereof). In particular, the present invention provides amphiphilic polymer compositions and methods of using the compositions to solubilize, enrich and isolate components of biological membranes, including membrane proteins, while retaining function and/or activity of the component.

Accordingly, in some embodiments, the present invention provides an amphiphilic polymer composition comprising negatively charged and/or neutral amphiphilic polymers. The polymers find use in the isolation and purification of membrane components (e.g., proteins or protein lipid complexes), while maintaining function and/or activity of the proteins. The isolated or purified membrane proteins of the present invention find use in diagnostic, research, and drug screening applications. The methods and compositions of the present invention find use with a variety of membrane proteins with diagnostic, therapeutic and research utility such as signaling proteins (e.g., G-protein coupled receptors (GPCRs)), drug metabolizing enzymes (e.g., Cytochrome P450s and UGTs) and other membrane proteins, including, but not limited to, membrane-associated proteins, transmembrane proteins, membrane transporters, ion channels, glycoproteins and membrane-associated enzymes or enzyme domains. The present invention is not limited by the nature of the protein.

For example, in some embodiments, the present invention provides a composition comprising a first negatively charged amphiphilic polymer and/or a second neutral amphiphilic polymer. In some embodiments, the first amphiphilic polymer is a phospholipid-PEG conjugate. In certain embodiments, the phospholipid comprises first and second hydrocarbon chains, and wherein the first and second hydrocarbon chains are between 10 and 20, and preferably betweeen 14 and 18 carbons in length. In some embodiments, the first hydrocarbon chain is a different length than the second hydrocarbon chain. In some preferred embodiments, the first and second hydrocarbon chains are saturated. In other embodiments, the first and second hydrocarbon chains are partially unsaturated. In some embodiments, the second amphiphilic polymer is a diacylglycerol-PEG conjugate. In some embodiments, the diacylglycerol comprises first and second hydrocarbon chains, wherein the first and second hydrocarbon chains are between 10 and 20, and preferably between 14 and 18 (e.g., 14, 16, or 18) carbons in length. In some embodiments, the first hydrocarbon chain is a different length than the second hydrocarbon chain. In some preferred embodiments, the first and second hydrocarbon chains are saturated. In other embodiments, the first and second hydrocarbon chains are partially unsaturated. In some embodiments, at least one of the polymers is covalently linked to a tag (e.g., an affinity tag (e.g., biotin, GST, His tag, or maltose binding protein), a label (e.g., a fluoresent label) or other tag). In some embodiments, the first and/or second polymers are provided at 0%, 10%, 20%, 30%, 40%, 50%, 60%, 70%, 80%, 90%, or 100% of the mixture (it should be understood that any value between these percentages is also contemplated).

In further embodiments, the present invention provides a kit for purification of membrane components (e.g., proteins or protein lipid complexes) comprising a composition comprising a first negatively charged amphiphilic polymer and/or a second neutral amphiphilic polymer as described herein. In some embodiments, the composition is configured for the full or partial solubilization, isolation, purification or enrichment of membrane proteins while maintaining activiy of the membrane proteins. In some embodiments, the kit further comprises instructions for using the kit for purifying membrane proteins. In other embodiments, the kit further comprises components for analyzing the concentration or activity of purified membrane proteins. In yet other embodiments, the kits comprise reagents for detecting the presence of membrane proteins (e.g., targeting proteins, antibodies, labeling reagents and other reagents). In still further embodiments, the kit further comprises components for purifying membrane proteins on a solid support. In some embodiments, the kits further comprise capture reagents (e.g., biotin), affinity tags or antibodies. In yet other embodiments, the kit further comprises components for performing a drug screening assay with purified membrane proteins. In still further embodiments, the present invention provides kits for formulating a membrane protein for delivery to a cell (e.g., a cell in an animal). In some embodiments, at least one of the polymers is covalently linked to a tag (e.g., an affinity tag (e.g., biotin, GST, His tag, or maltose binding protein), a label (e.g., a fluoresent label) or other tag). In some embodiments, the kit further comprises an additional membrane solubilization reagent (e.g., a detergent).

The present invention also provides a method, comprising: providing a membrane component containing sample, wherein the sample comprises a mixture of membrane components (e.g., proteins) which include the membrane component of interest; and isolating the membrane component using a composition comprising a first negatively charged amphiphilic polymer and/or a second neutral amphiphilic polymer as described above. In some embodiments, the membrane is a cell membrane, a organelle membrane or a viral membrane. In some preferred embodiments, the membrane protein is an enzyme. In some embodiments, the enzyme retains activity after the isolating (e.g., 95%, 90%, 80%, 70%, 60%, 50% of the activity observed in an un-isolated form). In some embodiments, the membrane protein of interest is purified (e.g., greater than 20%, preferably greater than 30% and even more preferably greater than 40% separated from the components with which it is normally found), and preferably substantially purified (e.g., greater than 60%, preferably greater than 80% and even more preferably greater than 90% or 95% and even more preferably 99% separated from the components with which it is normally found, after the isolating. In some embodiments, the specific activity of the protein is hight following the isolation relative the the specific activty prior to the isolating. In some embodiments, prior to the isolating step, the method further comprises the step of separating cell membrane fractions and washing the separated cell membrane fractions. In some embodiments, the membrane protein is a drug metabolizing enzyme (e.g., a cytochrome P450 enzyme or a glucuronidation enzyme). In other embodiments, the membrane protein is a G protein coupled receptor. In yet other embodiments, the membrane protein is selected from the group including, but not limited to, receptor guanylyl cyclases; receptor tyrosine kinases (e.g., EGF receptor); protein tyrosine phosphatases (e.g., CD45); integrins (alpha, beta chains); cadherins (e.g., E-cadherin); chemotaxis receptors; potassium channels (e.g., Kcs K channel); connexins; photosynthetic reaction center (e.g., L, M subunits); ABC transporters; voltage-gated K⁺ channels (e.g., shaker); G-coupled receptors (e.g., transducin, chemokine receptors, acetylcholine receptor; ion pumps (e.g., Ca⁺⁺ pump catalytic subunit, Na⁺K⁺ pump catalytic sub); CIC channels (e.g., CIC-1 of skeletal muscle); ABC transporters (e.g., MDR ATPase, peptide pump, CFTR); anion transporters (e.g., Band 3 protein). In some embodiments, the method further comprises the step of analyzing the activity of the isolated membrane protein of interest. In certain embodiments, the isolating is performed on a solid support. In some embodiments, at least one of the polymers is covalently linked to a tag (e.g., an affinity tag (e.g., biotin, GST, His tag, or maltose binding protein), a label (e.g., a fluoresent label) or other tag).

In some embodiments, the method further comprises the step of contacting the isolated membrane protein of interest with a test compound and measuring the level of activity in the presence and absence of the test compound (e.g., a drug).

The present invention further provides a method of analyzing membrane proteins, comprising: isolating a plurality of membrane proteins using the method described herein; generating a microarray of the plurality of membrane proteins; and measuring the activity of the plurality of membrane proteins. In some embodiments, the plurality of membrane proteins are unique variants of a membrane protein. In some embodiments, the variants are the result of single nucleotide polymorphisms in a gene encoding the variants. In some embodiments, the membrane proteins are drug metabolizing enzymes (e.g., UGT1A1 or variants of UGT1A1). In some embodiments, the method further comprises the step of contacting said microarray with one or more test compounds (e.g., drugs) and determining the activity of the proteins on the microarray in the presence and absence of the test compounds. The present invention is not limited by the nature of the membrane from which the membrane proteins of interest are isolated. For example, in some embodiments the membranes are cell membranes, mitochondrial membranes, endoplasmic reticulum membranes, plant or bacterial or viral membranes.

### DESCRIPTION OF THE FIGURES

FIG. 1 shows the activity of UGT1A1 following extraction with one exemplary reagent of the present invention (PRESERVEX-QML).

FIG. 2 shows the activity of motilin receptor, a G protein coupled receptor, after solubilization with one exemplary reagent of the present invention.

FIG. 3 shows the stability of BODIPY-motilin in exemplary lipid compositions of the present invention.

FIG. 4 shows the stability of COX2 over time in exemplary lipid compositions of the present invention.

FIG. 5 shows the results of a competitive binding experiment with the estrogen receptor in exemplary lipid compositions of the present invention.

FIG. 6 shows a reduction of light scattering in CYP3A4 Baculosomes in exemplary lipid compositions of the present invention.

FIG. 7 shows the relative fluorescence of two different solutions containing UGT 1A1 and in exemplary lipid compositions of the present invention over a 15 minute time interval.

FIG. 8 shows the stability of UGT 1A1 at room temperature for 48 hours in exemplary lipid compositions of the present invention.

FIG. 9 shows the binding affinity of UGT1A1 treated with lipid compositions of the present invention.

FIG. 10 shows that addition of a mixture of phospholipid-PEG and di-stearolglycerol-PEG increases assay tolerance to DMSO.

FIG. 11 shows the effects of using a mixture of lipid formulations on compound potency.

FIG. 12 shows the effects of using a mixture of lipid formulations on compound potency.

FIG. 13 shows the effects of using a mixture of lipid formulations on compound potency.

### DEFINITIONS

To facilitate understanding of the invention, a number of terms are defined below.

As used herein, the term “amphiphilic polymer” refers to polymeric molecules having a polar water-soluble polymeric chain attached to a hydrocarbon. As used herein, the term “negatively charged amphiphilic polymer” refers to an amphiphilic polymer with a net negative charge (e.g., the polymer comprises more positively charged moieties than negatively charged moieties). As used herein the term “neutral amphiphilic polymer” refers to an amphiphilic polymer with a net charge of zero.

As used herein, the term “hydrocarbon chain” refers to greater than two carbon atoms linked by single, double, or triple bonds. As used herein, the term “saturated hydrocarbon chain” refers to a hydrocarbon chain where all of the carbons are linked with single bonds. As used herein, the term “unsaturated hydrocarbon chain” or “partially unsaturated hydrocarbon chain” refers to a hydrocarbon chain with one or more double or triple carbon-carbon bonds.

As used herein, the terms “solid support,” “solid surface,” “support,” or “surface” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads or microspheres). Materials attached to a solid support may be attached to any portion of the solid support (e.g., may be attached to an interior portion of a porous solid support material). Preferred embodiments of the present invention have biological molecules such as nucleic acid molecules and proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the terms “bead,” “particle,” and “microsphere” refer to small solid supports that are capable of moving about in a solution (i.e., have dimensions smaller than those of the enclosure in which they reside). In some preferred embodiments, beads are completely or partially spherical or cylindrical. However, beads are not limited to any particular three-dimensional shape.

As used herein, the term “microarray” refers to a solid support with a plurality of molecules (e.g., nucleotides, peptides, etc.) bound to its surface. Microarrays, for example, are described generally in Schena, “Microarray Biochip Technology,” Eaton Publishing, Natick, Mass., 2000. Additionally, the term “patterned microarrays” refers to microarray substrates with a plurality of molecules non-randomly bound to its surface.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “genome” refers to the genetic material (e.g., chromosomes) of an organism.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length protein or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. Amino acid sequences can comprise naturally occurring or non-natural amino acids (e.g., amino acids not found in nature).

As used herein, the term “selectable marker” refers to a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene which is used in conjunction with CAD-deficient cells and the mammalian hypoxanthine-guanine phosphoribosyl transferase (hprt) gene which is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “response,” when used in reference to an assay, refers to the generation of a detectable signal (e.g., accumulation of reporter protein, increase in ion concentration, accumulation of a detectable chemical product).

As used herein the term “membrane proteins” refers to any protein that spans or is associated with a cell, organelle or viral membrane. In some embodiments, membrane proteins are integral membrane proteins that span the membrane (e.g., 7 trans membrane domain receptors). In other embodiments, membrane proteins are membrane associated proteins (e.g., proteins with an amphipathic alpha helix). In some embodiments, membrane proteins are anchored to the membrane by hydrophobic regions rather than transmembrane domains or by a covalently attached lipid or glycolipid (such as prenylated ras protein). Membrane protein may be associated with either surface of membranes. In other embodiments, membrane proteins are proteins that are not directly bound to membranes but exist in protein complexes where hydrophobic interactions facilitate binding (e.g., transcriptional complexes such as estrogen receptor.

As used herein, the term “membrane receptor proteins” refers to membrane spanning receptor proteins. Some membrane receptor proteins bind a ligand (e.g., a hormone or neurotransmitter). As is known in the art, protein phosphorylation is a common regulatory mechanism used by cells to selectively modify proteins carrying regulatory signals from outside the cell to the nucleus. The proteins that execute these biochemical modifications are a group of enzymes known as protein kinases. They may further be defined by the substrate residue that they target for phosphorylation. One group of protein kinases is the tyrosine kinases (TKs), which selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, epidermal growth factor (EGF), insulin, platelet-derived growth factor (PDGF), and fibroblast growth factor (FGF). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity (See, e.g., Ullrich and Schlessinger, Cell 61:203-212 [1990]). Tyrosine kinases can also be cytoplasmic, non-receptor-type enzymes and act as a downstream component of a signal transduction pathway.

As used herein, the term “signal transduction protein” refers to proteins that are activated or otherwise affected by ligand binding to a membrane receptor protein or some other stimulus. Examples of signal transduction protein include adenyl cyclase, phospholipase C, and G-proteins. Many membrane receptor proteins are coupled to G-proteins (i.e., G-protein coupled receptors (GPCRs); for a review, see Neer, 1995, Cell 80:249-257 [1995]). Typically, GPCRs contain seven transmembrane domains. Putative GPCRs can be identified on the basis of sequence homology to known GPCRs.

GPCRs mediate signal transduction across a cell membrane upon the binding of a ligand to a GPCR (e.g., to the extracellular portion). The intracellular portion of a GPCR interacts with a G-protein to modulate signal transduction from outside to inside a cell. A GPCR is therefore said to be “coupled” to a G-protein. G-proteins are composed of three polypeptide subunits: an α subunit, which binds and hydrolyses GTP, and a dimeric βγ by subunit. In the basal, inactive state, the G-protein exists as a heterotrimer of the α and βγ subunits. When the G-protein is inactive, guanosine diphosphate (GDP) is associated with the α subunit of the G-protein. When a GPCR is bound and activated by a ligand, the GPCR binds to the G-protein heterotrimer and decreases the affinity of the Gα subunit for GDP. In its active state, the G subunit exchanges GDP for guanosine triphosphate (GTP) and active Gα subunit disassociates from both the receptor and the dimeric βγ subunit. The disassociated, active Gα or βγ subunits transduce signals to effectors that are “downstream” in the G-protein signaling pathway within the cell. Eventually, the G-protein's endogenous GTPase activity returns Gα subunit to its inactive state, in which it is associated with GDP and the dimeric βγ subunit.

Numerous members of the heterotrimeric G-protein family have been cloned, including more than 20 genes encoding various Gα subunits. The various G subunits have been categorized into four families, on the basis of amino acid sequences and functional homology. These four families are termed Gαₛ, Gαᵢs, Gα_{q}, and Gα₁₂. Functionally, these four families differ with respect to the intracellular signaling pathways that they activate and the GPCR to which they couple.

For example, certain GPCRs normally couple with Gαₛ and, through Gαₛ, these GPCRs stimulate adenylyl cyclase activity. Other GPCRs normally couple with Gα_{q}, and through Gα_{q}, these GPCRs can activate phospholipase C (PLC), such as the β isoform of phospholipase C (i.e., PLCβ, Stermweis and Smrcka, Trends in Biochem. Sci. 17:502-506 [1992]).

As used herein, the term “protein kinase” refers to proteins that catalyze the addition of a phosphate group from a nucleoside triphosphate to an amino acid side chain in a protein. Kinases comprise the largest known enzyme superfamily and vary widely in their target proteins. Kinases may be categorized as protein tyrosine kinases (PTKs), which phosphorylate tyrosine residues, and protein serine/threonine kinases (STKs), which phosphorylate serine and/or threonine residues. Some kinases have dual specificity for both serine/threonine and tyrosine residues. Almost all kinases contain a conserved 250-300 amino acid catalytic domain. This domain can be further divided into 11 subdomains. N-terminal subdomains I-IV fold into a two-lobed structure that binds and orients the ATP donor molecule, and subdomain V spans the two lobes. C-terminal subdomains VI-XI bind the protein substrate and transfer the gamma phosphate from ATP to the hydroxyl group of a serine, threonine, or tyrosine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue required for maximal catalytic activity, and subdomains VI through IX comprise the highly conserved catalytic core. STKs and PTKs also contain distinct sequence motifs in subdomains VI and VIII, which may confer hydroxyamino acid specificity. Some STKs and PTKs possess structural characteristics of both families. In addition, kinases may also be classified by additional amino acid sequences, generally between 5 and 100 residues, which either flank or occur within the kinase domain.

Non-transmembrane PTKs form signaling complexes with the cytosolic domains of plasma membrane receptors. Receptors that signal through non-transmembrane PTKs include cytokine, hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (See, e.g., Carbonneau, H. and Tonks, Annu. Rev. Cell Biol. 8:463-93 [1992]). Regulation of PTK activity may therefore be an important strategy in controlling some types of cancer.

Examples of protein kinases include, but are not limited to, cAMP-dependent protein kinase, protein kinase C, and cyclin-dependent protein kinases (See, e.g., U.S. Pat. Nos. 6,034,228; 6,030,822; 6,030,788; 6,020,306; 6,013,455; 6,013,464; and 6,015,807, each of which is incorporated herein by reference).

As used herein, the term “protein phosphatase” refers to proteins that remove a phosphate group from a protein. Protein phosphatases are generally divided into two groups, receptor and non-receptor type proteins. Most receptor-type protein tyrosine phosphatases contain two conserved catalytic domains, each of which encompasses a segment of 240 amino acid residues (See e.g., Saito et al., Cell Growth and Diff. 2:59 [1991]). Receptor protein tyrosine phosphatases can be subclassified further based upon the amino acid sequence diversity of their extracellular domains (See e.g., Krueger et al., Proc. Natl. Acad. Sci. USA 89:7417-7421 [1992]). Examples of protein phosphatases include, but are not limited to, cdc25 a, b, and c, PTP20, PTP1D, and PTPλ (See e.g., U.S. Pat. Nos. 5,976,853; 5,994,074; 6,004,791; 5,981,251; 5,976,852; 5,958,719; 5,955,592; and 5,952,212, all of which are incorporated herein by reference).

As used herein, the term “protein post-translational modification” refers to any modification of protein that occurs following translation. In some embodiments, lipid and glycolipid modifications cause proteins to be associated with membranes. Other modifications include, but are not limited to, phosphylation and artificial modifications that are done in vitro such as pegylation.

As used herein, the term “protein activity” refers to any activity of a protein including, but not limited to, enzymatic activity, ligand binding, drug transport, ion transport, protein localization, receptor binding, and structural activity. In preferred embodiments, proteins isolated using the compositions and methods of the present invention retain at least 10%, preferably at least 25%, more preferably at least 40%, still more preferably at least 60%, even more preferably at least 80%, and yet more preferably at least 90% of the activity of the protein in its native state. Protein activity may be assayed using any suitable method.

As used herein, the term “purified” refers to molecules, either nucleic or amino acid sequences that are removed from their natural environment isolated or separated. An “isolated amino acid sequence” is therefore a purified nucleic acid sequence. “Purified amino acids” are at least 10%, preferably at least 20%, more preferably at least 25%, still more preferably at least 30%, even more preferably at least 40%, and yet more preferably at least 50% removed from other components with which they are naturally associated. “Substantially purified” molecules are at least 55% free, preferably at least 60% free, even more preferably at least 70% free, still more preferably at least 75% free, yet more preferably at least 80% free, yet more preferably at least 90% free, still more preferably at least 95%, and even more preferably at least 99% free from other components with which they are naturally associated. In some embodiments, isolated or purified amino acids have low purity but exhibit increased activity relative to non-isolated or un-purified amino acids.

As used herein, the term “tag” refers to any molecule attached to a polymer of the present invention. Examples of tags include, but are not limited to, proteins (e.g., containing natural or non-natural amino acids), labels, affinity tags (e.g., His tag, GST, maltose binding protein etc), and antibodies.

The term “label” as used herein refers to any atom or molecule that can be used to provide a detectable (preferably quantifiable) effect, and that can be attached to biological molecule (e.g., a nucleic acid, protein or lipid). Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress (“quench”) or shift emission spectra by fluorescence resonance energy transfer (FRET).

The term “test compound” refers to any chemical entity, pharmaceutical (e.g., small molecule or protein (e.g., antibody)), drug, and the like contemplated to be useful in the treatment and/or prevention of a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

### DETAILED DESCRIPTION OF THE INVENTION

The present invention provides methods and compositions for the extraction of membrane proteins. In particular, the present invention provides amphiphilic polymer compositions and methods of using the compositions to extract membrane proteins while retaining membrane protein activity.

Currently available products for isolating membrane proteins include polymeric surfactants (e.g., available from Anatrace, Maumee, Ohio) and Nanodiscs (available from Nanodisc, Ill.). However, these products have several disadvantages that make them poor choices for purification of membrane proteins. For example, many currently used detergents require purification of proteins before solubilization. Other products cut holes or sections of membranes.

In contrast, the methods of the present invention require no lengthy prior purification steps and do not disrupt membrane integrity. In some embodiments, the compositions and methods of the present invention extract protein-lipid complexes. The present invention is not limited to a particular mechanism. Indeed, an understanding of the mechanism is not necessary to practice the present invention. Nonetheless, it is contemplated that by maintaining intact protein-lipid (e.g., membrane) complexes, that activity of the protein is maintained. Accordingly, the compositions and methods of the present invention provide advantages over existing technologies including the ability to isolate membrane proteins, while retaining their function and/or activity. Exemplary compositions of the present invention and methods for their use are described below.

I. Amphiphilic Compositions

In some embodiments, the present invention provides amphiphilic compositions comprising one or more (e.g., a mixture) of amphiphilic polymers. In preferred embodiments, the compositions of the present invention comprise a mixture of negatively charged polymers and/or neutral polymers.

For example, in some embodiments, the present invention provides a composition comprising a first negatively charged amphiphilic polymer and/or a second neutral amphiphilic polymer. In some embodiments, the first amphiphilic polymer is a phospholipid-PEG conjugate. In certain embodiments, the phospholipid comprises first and second hydrocarbon chains, and wherein the first and second hydrocarbon chains are between 10 and 20, and preferably betweeen 14 and 18 carbons in length. In some embodiments, the first hydrocarbon chain is a different length than the second hydrocarbon chain. In some preferred embodiments, the first and second hydrocarbon chains are saturated. In other embodiments, the first and second hydrocarbon chains are partially unsaturated. In some embodiments, the second amphiphilic polymer is a diacylglycerol-PEG conjugate. In some embodiments, the diacylglycerol comprises first and second hydrocarbon chains, wherein the first and second hydrocarbon chains are between 10 and 20, and preferably between 14 and 18 (e.g., 14, 16, or 18) carbons in length. In some embodiments, the first hydrocarbon chain is a different length than the second hydrocarbon chain. In some preferred embodiments, the first and second hydrocarbon chains are saturated. In other embodiments, the first and second hydrocarbon chains are partially unsaturated. In some embodiments, at least one of the polymers is covalently linked to a tag (e.g., an affinity tag (e.g., biotin, GST, His tag, or maltose binding protein), a label (e.g., a fluoresent label) or other tag). In some embodiments, the first and/or second polymers are provided at 0%, 10%, 20%, 30%, 40%, 50%, 60%, 70%, 80%, 90%, or 100% of the mixture (it should be understood that any value between these percentages is also contemplated).

In some embodiments, the negatively charged amphiphilic polymer is a phospholipid-polyethylene glycol (PEG) conjugate. The present invention is not limited to a particular lipid. Any suitable lipid may be utilized in the phopholipid-PEG conjugates of the present invention. In some embodiments, the lipids contain hydrocarbon tails of various lengths (e.g., the same phospholipid comprises two hydrocarbon chains of different lengths). In some preferred embodiments, hydrocarbon chains range from 10 to 20 carbons. In particularly preferred embodiments, hydrocarbon chains range from 14 to 18 carbons. In preferred embodiments, saturated or partially unsaturated hydrocarbon tails are preferred.

In certain embodiments, the neutral amphiphilic polymer is a diacylglycerol-PEG conjugate. The present invention is not limited to particular acyl chains. In some embodiments, the diacylglycerol-PEG conjugates comprise hydrocarbon tails of various lengths (e.g., the same compound comprises two hydrocarbon chains of different lengths). In some preferred embodiments, hydrocarbon chains range from 10 to 20 carbons. In particularly preferred embodiments, hydrocarbon chains range from 14 to 18 carbons. In some even more preferred embodiments, stearoyl (18 carbon), palmitoyl (16 carbon) or miristoyl (14 carbon) hydrocarbon chains are preferred. In preferred embodiments, saturated or partially unsaturated hydrocarbon tails are preferred.

In some embodiments, the phospholipids is phosphatidylethanolamine. In some embodiments, PEG is attached via the amino group in the phospholipid's polar head. In other embodiments, acrylic based polymers (e.g., polyacrylates) are utilized as amphiphilic polymers. In some embodiments utilizing neutral diacylglycerols, the PEG chain is attached via a free glycerol hydroxyl. The present invention is not limited to a particular molecular weight of PEG. In some preferred embodiments, a molecular weight of 2000 is an optimal for the PEG chain.

The present invention is not limited to the use of phospholipids. Any other amphiphilic block-copolymers can serve as a suitable alternative to phospholipids-PEG and diacylglycerol-PEG conjugates. In preferred embodiments, the length of the hydrophobic block is consistent with the thickness of lipid bilayer. Examples of hydrophobic chains include, but are not limited to, polymethyl- and polyetylacrylates, polystyrenes and other hydrophobic vinyl polymers, polyesters, such as polylactides and polyglycolides, and hydrophobic polyamides. Examples of suitable hydrophilic blocks include, but are not limited to, polyacrylamides, polyacrylates, and polyacrylic esters.

In preferred embodiments, the molar percent of the negatively charged amphiphilic composition is chosen to mimic the amount of negatively charged lipids in mammalian cell membranes. In some embodiments, the critical micelle concentration (CMC) of the amphiphilic compositions of the present invention varies based on the hydrocarbon side chains of the conjugates. In preferred embodiments, the CMC is optimized for the particular application. The optimal ratio of neutral to negatively-charged polymers in micellar preparations is dependent on the specific membrane protein to be extracted. In some embodiments, a ratio of 90% (mol) of neutral component/10% (mol) negatively-charged component is utilized.

In some further embodiments, the compositions comprise a mixture of neutral amphiphilic polymers and amphiphilic polymers (negatively-charged or neutral) carrying an affinity tag (e.g., biotin).

It should be understood that the present invention is not limited to the particular components described above. Any component or combination of components may be used so long as it: forms an amphiphilic complex; is capable of isolating membrane components (proteins, peptides, carbohydrates, lipids, or combinations thereof); and is capable of retaining desired biological properties (e.g., enzyme activity) of the isolated component. Other desired properties include shelf-life, lack of toxicity, function under a range of temperatures, etc.

II. The Present Invention in Use

As described above, in some embodiments, the present invention provides amphiphilic polymers for use in isolating membrane proteins. The compositions of the present invention have been demonstrated to isolate membrane proteins while maintaining protein activity (See e.g., experimental section below). The isolated membrane proteins find use in a variety of research and clinical applications including, but not limited to, those described below.

A. Isolation of Membrane Components

The amphiphilic compounds of the present invention find use in the isolation of membrane proteins. While the below description utilizes membrane proteins as an exemplary use for the compositions and methods of the present invention, the compositions and methods of the present invention are further suitable for isolation of additional membrane components (e.g., lipids). An exemplary isolation protocol is provided in the experimental section below. For example, in some embodiments, cells are lysed and the membranes are centrifuged into a pellet. The membrane fraction is then resuspended in a composition of the present invention. In some embodiments, the solubilized membrane fractions are separated from the insoluble fraction. In some embodiments, the compositions of the present invention are used in combination with an additional membrane solubilization reagent such as a detergent.

Membrane preparation using the compositions of the present invention results in the isolation of intact, active membrane fractions. In some embodiments, the membrane proteins are purified (e.g., at least 10%, preferably at least 20%, more preferably at least 25%, still more preferably at least 30%, even more preferably at least 40%, and yet more preferably at least 50% removed from other components with which they are naturally associated. In other embodiments, the membrane proteins are substantially purified (e.g., at least 55% free, preferably at least 60% free, even more preferably at least 70% free, still more preferably at least 75% free, yet more preferably at least 80% free, yet more preferably at least 90% free, still more preferably at least 95% free from other components of the cell with which they are naturally associated). In some embodiments, it is preferred that membrane proteins be purified, but not substantially purified. The present invention is not limited to a particular mechanism. Indeed, an understanding of the mechanism is not necessary to practice the present invention. However, it is contemplated that use of the amphiphilic compositions of the present invention results in the isolation of intact membrane fragments in which a membrane protein of interest and any associated proteins or cofactors remain associated with the membrane protein during purification. Thus, a membrane protein is unlikely to be separated from other cofactors or cellular components necessary for optimum activity of the membrane protein.

In some preferred embodiments, isolated membrane proteins retain the activity or function of the membrane protein. In some embodiments, at least 10%, 15%, or 20%, and preferably at least 25%, 30%, 40, or 50%, and even more preferably at least 60%, 70%, or 75%, and still more preferably at least 80%, 90%, or 95% of the activity of the un-isolated membrane protein is retained. Activity and/or function of the membrane protein may be assayed using any suitable method. In some embodiments, enzyme assays with the appropriate substrates for the membrane protein of interest are utilized. In other embodiments, binding of ligand or substrates is assayed (e.g., using any suitable assay).

In some embodiments, the membrane protein isolation methods of the present invention are performed in solution. In other embodiments, they are performed on a solid support. Any suitable solid support or surface that the amphiphilic compositions of the present invention can be attached to may be utilized in the methods of the present invention including, but not limited to, column supports, microtitre plates, and slides.

In some embodiments, following isolation of membrane proteins using the amphiphilic compositions of the present invention, membrane proteins are further fractionated or purified. Any known protein purification method that is compatible with the amphiphilic compositions of the present invention may be utilized. For example, in some embodiments, methods that separate proteins based on size (e.g., size exclusion chromatography or native get electrophoresis) are utilized. In other embodiments, affinity tags attached to the reagents of the present invention are used to purify membrane proteins.

In some embodiments, compositions of the present invention are added to a concentrated protein solution and upon dilution, the amount of protein activity is increased (e.g., due solubilization of the protein). In other embodiments, compositions of the present invention are used in protein partitioning (e.g., in combination with dextrans or PEGS and propylene oxides) to separate proteins in solution.

In other embodiments, the compositions of the present invention are used to stabilize membrane proteins or protein lipid complexes that have been isolated by other methods (e.g., detergents). For example, in some embodiments, membrane proteins or protein lipid complexes are first isolated using conventional methods such as detergents. Following isolation, a composition of the present invention is added to the protein or protein lipid complex. It is contemplated that the addition of a composition of the present invention will increase stability of the protein and thus increase or maintain activity.

The methods and compositions of the present invention are suitable for use in the purification of any membrane protein. Experiments conducted during the course of development of the present invention resulted in the isolation of active UGT1A1, Cyclooxygenase-2 (COX-2), and cytochrome P450 enzymes using the compositions and methods of the present invention. The present invention is not limited to the purification of a particular membrane protein.

The list of integral membrane proteins, sometimes also referred to as transmembrane proteins, is vast. Transmembrane proteins may cross the membrane only once or over twenty times. Many transmembrane proteins associate with other transmembrane proteins to form larger complexes. Such complexes may be comprised of two identical subunits (such as homodimers) or two different protein subunits (such as heterodimers). There are examples of even larger complexes of three (sodium ion channel, Na⁺/K⁺ ATPase), four (aquaporin), five (cation channels of nicotinic receptors, anion channels of glycine receptors) or more (photoreaction center, mitochondrial respiratory chain) homologous or heterologous subunits.

Transmembrane proteins contribute to a wide variety of cellular functions, including the transport of molecules and ions into or out of cells, cell recognition, cell-to-cell communication, and cell adhesion. One simple way to classify transmembrane proteins is by their number of transmembrane domains.

The group of transmembrane proteins that only cross the membrane once (also known as single-pass proteins) is particularly diverse both structurally and functionally. This class includes a large number of cell surface receptor proteins. For example, the EGF receptor binds epidermal growth factor, which leads to activation of the receptor's tyrosine kinase activity. Other examples of single-pass transmembrane proteins include the integrins and cadherins, which function in cell-cell communication via binding to extracellular molecules.

Another large class of cell surface receptors is the G-protein coupled receptors (GPCRs), which span the membrane seven times. Unlike many of the single-pass receptors, these proteins do not have enzymatic activity themselves but instead are functionally linked to signaling proteins known as G proteins. The chemokine receptor CCR5 that serves as the principal coreceptor for HIV-1 is a typical example of a G protein-coupled receptor. Other well studied members of this class include transducin, which senses light, and the acetylcholine receptor, which binds neurotransmitter at neuronal synapses.

Because of its hydrophobic interior, the plasma membrane is highly impermeant to most polar molecules including small molecules such as ions, sugars, amino acids, nucleotides, and many cell metabolites. Membrane transport proteins fall into two general classes: a) carrier proteins, which bind the specific solute to be transported and undergo a conformational change to allow its transit, and b) channel proteins, which allow specific solutes, most often inorganic ions, to cross the membrane when they are open and form a channel.

Well-studied carrier proteins include the ABC transporters (spanning the membrane 6 times), which bind solute as well as ATP and change conformation upon the hydrolysis of ATP to ADP. Many ion pumps are examples of gated carrier proteins, such as the 10-membrane spanning catalytic subunit of the calcium pump.

Ions also cross membranes in channel proteins, which are typically gated so that they only open in response to a specific signal (such as a change in membrane voltage). Examples include some potassium channels (e.g. the Kcs K⁺ channel), which spans the membrane twice, and voltage-gated potassium channels such as the Drosophila Shaker protein (spanning the membrane 6 times).

In other embodiments, the transmembrane proteins are envelope proteins (e.g., lentiviral proteins). The lentiviral proteins can include, for example, proteins from human immunodeficiency virus (HIV) (e.g., HIV-1 gp120 or HIV-1 gp160), feline immunodeficiency virus (FIC), or visna virus.

Other examples of viral envelope proteins include, for example, envelope proteins from filoviruses (e.g., Ebola virus), orthomyxoviruses (e.g., influenza virus), VSV-G, alpha viruses (e.g., Semliki forest virus and Sindbis virus), arena viruses (e.g., lymphocytic choriomeningitis virus), flaviviruses (e.g., tick-borne encephalitis virus and Dengue virus), rhabdoviruses (e.g., vesicular stomatitis virus and rabies virus), Moloney leukemia virus, HSV, VZV, Mumps virus, Rhinoviruses, Measles, Rubella, Arbovirus, Enteroviruses (e.g., Polio, Coxsackie, Echoviruses), Coxsackie B, A and Echovirus, Rhinoviruses, Hepatitis viruses, Norwalk virus, Astroviruses, Togavirus, Alphaviruses, Pestiviruses, Coronavirus, Parainfluenza, Mumps virus, Measles virus, Respiratory Syncytial Virus (RSV), Bunyaviridae, Reoviridue, Reoviruses, Rotaviruses, HTLV, Polyomaviruses, Papillomaviruses, Adenoviruses, Parvoviruses, EBV, CMV, Varicella Zoster virus, herpes viruses, and Pox viruses.

Further examples of membrane proteins include, but are not limited to, Receptor guanylyl cyclases (e.g., Sperm React receptor); receptor tyrosine kinases (e.g., EGF receptor); protein tyrosine phosphatases (e.g., CD45); integrins (alpha, beta chains); cadherins (e.g., E-cadherin); chemotaxis receptors; potassium channels (e.g., Kcs K channel); connexins; photosynthetic reaction center (e.g., L, M subunits); ABC transporters; voltage-gated K⁺ channels (e.g., shaker); G-coupled receptors (e.g., Transducin, Chemokine receptors, Acetylcholine receptor; Ion pumps (e.g., Ca⁺⁺ pump catalytic subunit, Na⁺K⁺ pump catalytic sub); CIC channels (e.g., CIC-1 of skeletal muscle); ABC transporters (e.g., MDR ATPase, Peptide pump, CFTR); anion transporters (e.g., Band 3 protein).

B. Uses of Purified Membrane Proteins

Membrane proteins purified using the amphiphilic compositions of the present invention find use in any application in which purified, active membrane proteins are desired. The isolation methods of the present invention are compatible with direct measurement of phospholipid content (e.g., using a Bartlett assay), measurement of protein concentration (e.g., BCA or other assay), SDS-PAGE, immunoassays (e.g., ELISA and Western assays) and fluorescent detection at multiple wavelengths. In some embodiments, the purified membrane proteins of the present invention find use in drug screening, research, and diagnostic applications.

i. Diagnostic Applications

One illustrative example of a membrane protein, UGT1A1, that finds particular use with the methods and compositions of the present invention, is described in the Experimental section below and in the below description. The below description is illustrated with UGT1A1, an important and clinically relevant drug metabolizing enzyme. However, the methods and compositions of the present invention are suitable for use with any membrane protein.

Experiments conducted during the course of development of the present invention demonstrated the isolation of UGT1A1 from cell membranes with over 95% of its activity maintained.

Irinotecan is an important and currently available antineoplastic treatment. Irinotecan's chemical formula name is (S)-4,11-diethyl-3,4,12,14-tetrahydro-4-hydroxy-3,14-dioxyo-1H-pyranol[3′,4′:6,7]-indolizino[1,2-b]quinolin-9-y[1,4′-bipeperidine]-1′-carboxylate, monohydrochloride, trihydrate. The empirical formula for Irinotecan is C₃₃H₃₈N₄O₆.HCl.3H₂0 and has a molecular weight of 677.19. Irinotecan is currently sold under the name CAMPTOSAR by Pharmacia & Upjohn Corporation. Irinotecan is used to treat cancer (e.g., CAMPTOSAR is approved for colorectal cancer un the United States). The mechanism of action of Irinotecan and its active metabolite SN-38 is preventing topoisomerase I from functioning properly.

Irinotecan (also known as CPT-11) is transformed in vivo by carboxylesterases to an active metabolite called SN-38. SN-38 has about 100-1,000 fold higher antitumor activity than Irinotecan. Irinotecan has been shown to be metabolized by hepatic cytochrome P-450 3A enzymes to a compound called APC, which has a 500 fold weaker antitumor activity compared with SN-38. SN-38 is known to undergo significant bilary excretion and enterohepatic circulation. SN-38 is also subjected to glucuronidation by hepatic uridine diphosphate glucuronosyltransferases (UGTs) to form SN-38G. SN-38G is inactive and is excreted into the urine and bile. Failure to convert SN-38 to SN-38G has been suggested as a cause of diarrhea in patients administered Irinotecan due an accumulation of SN-38 (See, Lyer et al., J. Clin. Invest., 101 (4), Feb., 1998, 847-854, herein incorporated by reference).

Clinical studies have shown that Irinotecan was able to significantly improve tumor response rates, time to tumor progression and survival. Irinotecan has shown effectiveness when administered with 5-fluorouracil (5-FU) and leucovorin (LV). Irinotecan is generally administered intravenously.

There are many side effects associated with Irinotecan therapy. One side effect is cholinergic symptoms (e.g. early-onset diarrhea, contraction of pupils, lacrimation, flushing, rhinitis, increased salivation, diaphoresis, and abdominal cramping). Administration of atropine is generally recommended to counteract these symptoms. Another known side effect is late-onset diarrhea, which may be treated with loperamide, IV hydration, and oral antibiotics). Another known side effect is nausea and vomiting. Administration of antiemetic agents on the day of Irinotecan treatment may be used to counteract nausea and vomiting. Finally, another Irinotecan side effect is severe myelosuppression, with deaths due to sepsis being reported.

UGTs are microsomal enzymes catalyzing the glucuronidation of numerous endogenous and exogenous substrates. Glucuronidation increases the polarity of the substrates to allow them to be better eliminated from the body. The human UGTs are classified into UGT1 and UGT2 families. The UGT1 gene consists of at least 13 unique isoforms with variable exon 1 and common exons 2 to 5. Each of the exons 1 is preceded by its own promoter and differentially spliced to the common exons to produce a unique mature mRNA. The UGT1 family is further classified into multiple isoforms, i.e., UGT1A1, UGT1A3, UGT1A4, up to UGT1A12. The UGT1A1 isoform is responsible for the glucuronidation of bilirubin.

UGT1A1 polymorphism plays several roles in the metabolism of irinotecan. The example of irinotecan demonstrates how a polymorphism in an inactivating metabolic pathway may affect the therapeutic outcome in cancer chemotherapy. As described above, Irinotecan (CPT-11; 7-ethyl-10-[4-(1-piperidino)-1-piperidino]carbonyloxycamptothecin) is a camptothecin derivative used in the treatment of metastatic colorectal cancer. Irinotecan is a prodrug, since it needs to be activated by systemic carboxylesterases to SN-38 (7-ethyl-10-hydroxycamptothecin) in order to exert its antitumor activity mediated by the inhibition of topoisomerase I. SN-38 undergoes glucuronide conjugation to form the inactive SN-38 glucuronide (SN-38G; 10-O-glucuronyl-SN-38). In addition, two oxidated metabolites of irinotecan have been identified as APC (7-ethyl-10[4-N-(5-aminopentanoic acid)-1piperidino]carbonyloxycamptothecin) and NPC [7-ethyl-10-(4-amino-1-piperidino)carbonyloxycamptothecin] formed by CYP3A4 enzyme. APC and NPC have shown weak antitumor activity in vitro.

SN-38 has been associated with the severe diarrheal episodes occurring after irinotecan therapy as a result of the direct enteric injury caused by SN-38. Because it undergoes significant biliary excretion, SN-38 may potentially continue to remain in the gastrointestinal tract, resulting in prolonged diarrhea. The glucuronidation of SN-38 to the inactive SN-38G may protect against irinotecan-induced intestinal toxicities as a result of renal elimination of the more polar SN-38G.

The assessment of pharmacodynamics of SN-38 glucuronidation showed that, with respect to the total irinotecan available in the circulation, patients with relatively low glucuronidation rates had progressive accumulation of SN-38 leading to toxicity (Gupta et al., Cancer Res 54: 3723-3725, 1994). A genetic predisposition to the metabolism of irinotecan may be critical in patients with reduced UGT1A1 activity (Iyer et al., J Clin Invest 101:847-854, 1998, herein incorporated by reference). As the distinction between mild instances of the syndrome and normal condition is sometimes difficult, Gilbert's syndrome remains often undiagnosed.

Accordingly, in some embodiments, the present invention provides methods of screening individuals for their UGT1A1 or other membrane protein activity. For example, in some embodiments, UGT1A1 is purified from a cell sample using the methods and compositions of the present invention. UGT1A1 activity assays (e.g., metabolism of SN-38) are then used to compare an individual's UGT1A1 activity to normal and polymorphic controls. If an individual is found to have reduced UGT1A1 activity, the dosage of Irinotecan can be adjusted to avoid toxicity.

In certain embodiments, the sample comprising UGT1A1 is also screened with an assay to determine if the subject will benefit from a second drug that counteracts side-effects of Irinotecan administration (exampled of second drugs include, but are not limited to, atropine, loperamide, and antimetics). In other embodiments, the side effects are selected from early-onset diarrhea, contraction of pupils, lacrimation, flushing, rhinitis, increased salivation, diaphoresis, abdominal cramping, late-onset diarrhea, nausea, vomiting, myelosuppression, and sepsis. In certain embodiments, the subject is administered Irinotecan and a second drug to counteract the side effects of the Irinotecan administration.

Such methods find use in the diagnostic screening of other drug metabolizing enzymes (e.g., cytochrome P450 enzymes) and cyclooxygenase enzyme (e.g., COX-2). In some embodiments, samples are obtained from a patient's liver and the enzymes are isolated using the compositions and methods of the present invention. Activity assays are then performed on the isolated proteins. The direct measurement of activity of purified drug metabolizing membrane proteins finds use in the identification of individuals with altered drug metabolizing activity (e.g., over or under activity). Such information finds use in the customization of drug dosages. The proper dosage of a drug prevents toxicity from overdose in under metabolizing individuals and allows for increased dosages and effectiveness in under metabolizing individuals.

ii. Drug Screening Methods

In other embodiments, membrane proteins purified using the methods and compositions of the present invention find use in drug screening applications. For example, in some embodiments, signaling or drug metabolizing enzymes are purified using the methods and compositions described herein. The purified enzymes are then screened for their activity in the presence and absence of test compounds. In some embodiments, the membrane proteins are purified on solid support. In other embodiments, the compositions and methods are used to isolate membrane components for assaying drug absorption (e.g., using surface plasmon resonance (SPR)). In some embodiments, the invention may be used for drug absorption, distribution, metabolism, and excretion (ADME) analysis.

In some embodiments, the drug screening methods of the present invention are high-throughput drug screening methods. For example, in some embodiments, membrane proteins (e.g., drug metabolizing enzymes such as UGT1A1) are prepared using the methods and compositions of the present invention and then used to generate a protein microarray. In some embodiments, a series of polymorphic variants of a membrane protein (e.g., having altered activity) are generated and purified for use in protein microarrays. The microarray can then be screening for enzyme activity using known methods. Test compounds are added to the microarray and the effect of the test compounds on enzyme activity is assayed.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam, Anticancer Drug Des. 12:145 [1997]).

Examples of methods for the synthesis of molecular libraries can be found in the art, for example in: DeWitt et al., Proc. Natl. Acad. Sci. U.S.A. 90:6909 [1993]; Erb et al., Proc. Nat. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin, Science 249:404-406 [1990]; Cwirla et al., Proc. NatI. Acad. Sci. 87:6378-6382 [1990]; Felici, J. Mol. Biol. 222:301 [1991]).

iii. Research Applications

In still further embodiments, membrane proteins purified using the methods and compositions of the present invention find use in research applications. For example, in some embodiments, high throughput screening (e.g., using the protein microarrays described above or plate methods) is utilized to screen many variants of an enzyme (e.g., a drug metabolizing enzyme) concurrently. Membrane proteins purified using the methods and compositions of the present invention can also be screening for activity using standard (e.g., non-high throughput) methods known in the art. Any membrane protein (e.g., G-proteins, drug metabolizing enzymes and other signaling or receptor proteins) may be screened for activity using the methods and compositions of the present invention.

C. Kits

In some embodiments, the present invention provides kits for purifying and analyzing membrane proteins. For example, in some embodiments, the present invention provides kits for purifying membrane proteins using the amphiphilic compositions of the present invention. In some embodiments, the kits comprise an amphiphilic composition of the present invention (e.g., optimized for purification of a particular membrane protein or class of membrane proteins). In some embodiments, the kits further comprise instructions for using the amphiphilic compositions for purification of proteins. In certain embodiments, the kits further comprise additional reagents for use in purification of proteins (e.g., cell lysis or re-suspension buffers).

In some embodiments, the kits further comprise one or more additional components for use in the analysis or screening of purified membrane proteins. For example, in some embodiments, the kits of the present invention comprise reagents for determining protein concentration (e.g., BCA or other assay). In some embodiments, the kits comprise reagents for detecting the presence of membrane proteins, including, but not limited to, targeting proteins, antibodies, labeling reagents and other reagents.

In other embodiments, the kits comprise components for isolating or attaching membranes to a solid support such as including, but not limited to, beads, microtitre plates, columns, plates and other solid surfaces. In some embodiments, the kits further comprise capture reagents (e.g., avidin), affinity tags or antibodies.

In still further embodiments, the kits comprise reagents for performing activity assays (e.g., drug screening, diagnostic, or research assays). For example, in some embodiments, the kits comprise positive control proteins (e.g., membrane proteins with known activity), tracers, competitors, substrates, detection reagents (e.g., comprising a label), lipids and proteins for drug absorption assays, test compounds, and reagents for solubility testing. The present invention is not limited to the kit components described herein. One skilled in the art recognizes that additional reagents and instructions may be included depending on the desired application.

D. Drug Delivery

In yet other embodiments, the present invention provides methods and compositions for the delivery of drugs. For example, in some embodiments, drugs are encapsulated in the amphiphilic compositions of the present invention. Such methods are particularly well suited for the delivery of toxic drugs (e.g., chemotherapy agents such as TAXOL or TAXOTERE) to subjects.

### EXPERIMENTAL

The following examples serve to illustrate certain preferred embodiments and aspects of the present invention and are not to be construed as limiting the scope thereof. In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); and GTP (guanosine 5′-triphosphate).

### EXAMPLE 1

### Solubilization of UGT1A1 Membranes

A. Methods

UGT1A1 was isolated from Baculovirus-infected sf-9 cells expressing human UGT1A1. Cell membrane fractions were isolated using a standard protocol (McNamee et al., Biotechniques 7:465 [1989]). Membrane pellets were washed with HEPES buffered saline and microfuged. Protein concentration was measured using a BCA assay (Pierce Biotechnology, Rockford, Ill.). Washed membranes were resuspended in HEPES buffered saline at 0.5-4 mg/ml protein at 4° C. An amphiphilic polymer-based solubilization medium consisting of phospholipid-PEG conjugate and a di-stearolglycerol-PEG conjugate at a 1:20 protein:reagent (w/w) ratio was added and the solution was vortexed. The mixture was sonicated using a VWR Model 75D bath-type sonicator at maximum power for 30 seconds. The unsolubilized membrane proteins were precipitated in a centrifuge at 16000×g for 10 min at 4° C.

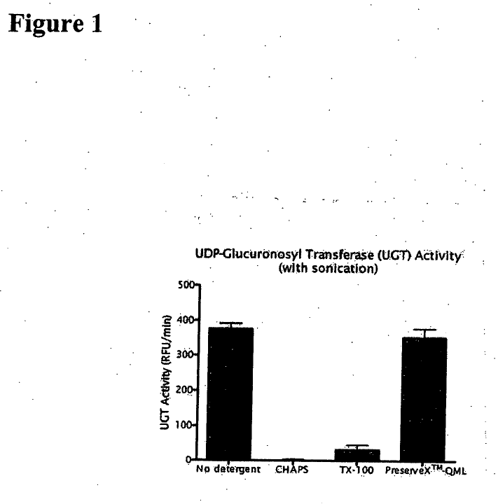

The supernatant was removed and analyzed for UGT1A1 activity. Over 95% of the activity was maintained during isolation (FIG. 1).

### EXAMPLE 2

### Solubilization of Human Motilin Receptor

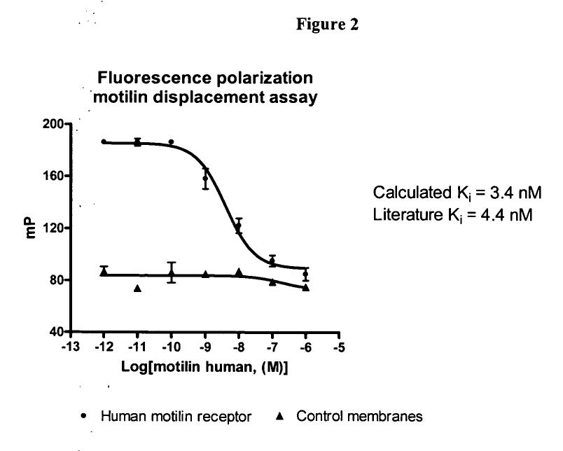

This example demonstrates that human motilin receptor, one of the G-protein-coupled receptors, can be incorporated into PreserveX polymeric micelles. Human motilin receptor membrane preparation (commercially available form PrerkinElmer Life and Analytical Sciences, Boston, Mass.) were mixed with phospholipid-PEG conjugate and a di-stearolglycerol-PEG conjugate (90/10 mixture) and were sonicated in 1:5 total protein to total polymer (w/w) ratio for 30 sec. Sonication conditions were identical to the above example. Fluorescence polarization displacement assay was performed with polymeric micelle-incorporated receptor with 6×10⁻¹⁰ M of BODIPY-TMR motilin as a tracer. The unlabeled motilin (Phoenix Pharmaceuticals, Belmont, Calif.) was used as a displacer in a range of concentrations (FIG. 2). FIG. 2 demonstrates that upon incorporation into the polymeric micelles the motilin receptor preserves full biological activity and demonstrates excellent specificity to its native ligand.

### EXAMPLE 3

### Stability of Fluorescent Ligand

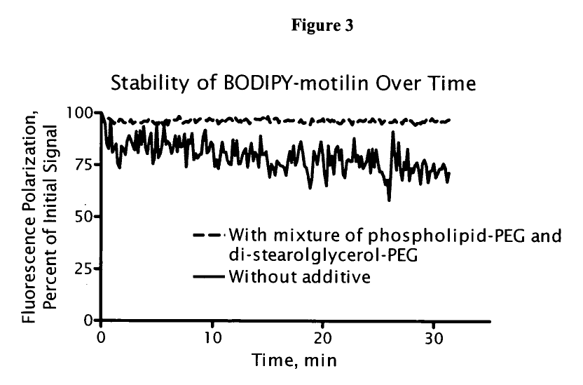

A mixture of phospholipid-PEG and di-stearolglycerol-PEG was mixed with BODIPY-motilin (a fluorescently-labeled 22 residue peptide; Perkin Elmer, Wellesely, Mass.), and fluorescence polarization was monitored continuously for 30 minutes. The use of a mixture of phospholipid-PEG and di-stearolglycerol-PEG resulted in a more stable signal (FIG. 3), which finds use in screening applications.

### EXAMPLE 4

### Stability of COX2 Over Time

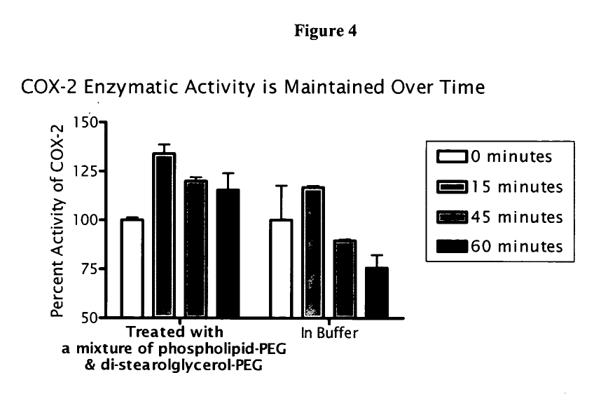

This Example shows that the enzymatic activity of cyclooxygenase-2 (COX-2; Sigma) is maintained when the protein is stabilized by a mixture of phospholipid-PEG and di-stearolglycerol-PEG. Activity of COX-2 was monitored with the Amplex® Red Colorimetric Assay (Invitrogen, Carlsbad, Calif.). The results are shown in FIG. 4.

### EXAMPLE 5

### Competitive Binding Experiment with Estrogen Receptor

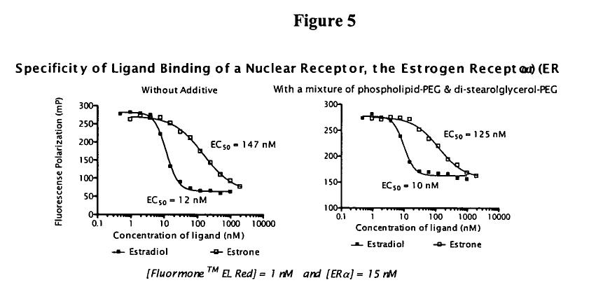

A mixture of phospholipid-PEG and di-stearolglycerol-PEG was mixed with estrogen receptor α (ERα; Invitrogen, Carlsbad, Calif.). Two natural ER ligands, estrone and estradiol, as well as tamoxifen citrate and 4-dehydroandrosterone (a negative control) were used in a competition assay with the fluorescent estrogen ligand Fluormone™ EL Red (Invitrogen). The results are shown in FIG. 5. The EC₅₀ values were similar for the control preparations as well as the samples treated with phospholipid-PEG and di-stearolglycerol-PEG mixture.

### EXAMPLE 6

### Reduction of Light Scattering in CYP3A4 Baculosomes

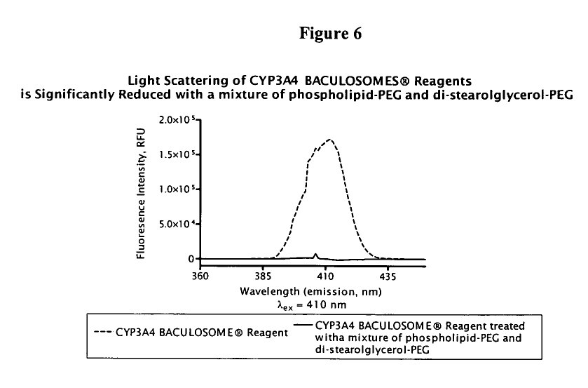

Experiments were performed in 20 mM HEPES buffer, pH 7.5, containing 5 nM recombinant human CYP3A4 (also containing human cytochrome b5, and rabbit NADPH-P450 reductase; Invitrogen) and a mixture of phospholipid-PEG and di-stearolglycerol-PEG. The enzymatic activity was monitored with the Vivid® Red Fluorometric Assay (Invitrogen). The signal was measured using a SpectraMax plate reader (Molecular Devices Corp., Sunnyvale, Calif.) at λₑₓ=410 nm. Control reactions were performed identically, except the control did not contain any PEG materials. The micellar CYP3A4 formulations exhibited a dramatic reduction in light scattering (FIG. 6).

### EXAMPLE 7

### UGT in High Throughput Screen

UGT1A1 Enzyme Preparations

UGT1A1 enzyme preparations were obtained as microsomal fractions of Sf9 cells infected with Baculovirus carrying insert for human UGT1A1. Prior to dispensing with robotic equipment, microsomal fractions were diluted 1:4 in HBS buffer, pH 7.5 containing a mixture of phospholipid-PEG and di-stearolglycerol-PEG and sonicated for 30″ using a bath-tub sonicator.

Reporter Substrate Preparations

7-hydroxy-6-methoxycoumarin glucuronidation was used as a reporter substrate reaction. Reporter substrate was diluted from a 100 mM stock solution prepared in methanol into HBS containing a mixture of phospholipid-PEG and di-stearolglycerol-PEG followed by sonication.

Assay Conditions

All assays were run in 384-well black microtiter plates (Cliniplate 384, Thermoelectron) in a total volume of 40 μl. The final concentrations were as follows: 50 mM HBS, pH 7.5 containing 2.5 mM UDPGA and a mixture of phospholipid-PEG and di-stearolglycerol-PEG, 0.7 mg/ml total protein, 10 mM 7-hydroxy-6-methoxycoumarin (reporter fluorescent substrate) and 10 mM UDPGA, a reaction co-factor. UDPGA was replaced with equal volume of water for the control preparations.

Fluorescent Readings

The fluorescent readings were obtained for 16 minutes at a room temperature in a kinetic mode using Tecan's Safire monochromator plate reader with excitation and emission wavelength set up at 410 and 590 nm correspondingly.

HTS Screening

All liquid transfers were performed using the Beckman Coulter Biomek FX liquid handler with a 384-channel disposable tip pipetting head. 352 compounds from UW HTS library collection were assayed using UGT1A1 stabilized with a mixture of phospholipid-PEG and di-stearolglycerol-PEG and control UGT1A1 preparations.

Results

Functional Assay for UGT 1A1 Stabilized with a Mixture of Phospholipid-PEG and Di-Stearolglycerol-PEG.

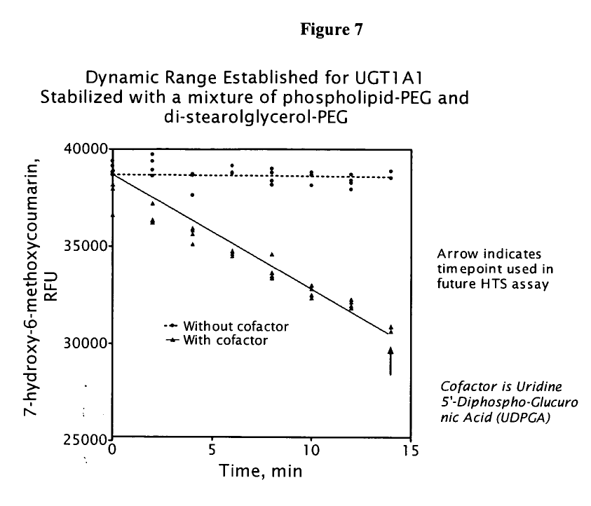

FIG. 7 shows the relative fluorescence of two different solutions over a 15 minute time interval. One solution contained 7-hydroxy-6-methoxycoumarin (7-h-6-m), which is natively fluorescent, mixed with UGT 1A1. The second solution contains the same materials as the first solution but also includes a cofactor of UDPGA. When the cofactor is added, the UGT 1A1 causes a sugar molecule to bind to the 7-h-6-m which then causes the fluorescent 7-h-6-m to lose fluorescence at a linear rate over time.

Stability of UGT 1A1 at Room Temperature for 48 Hours

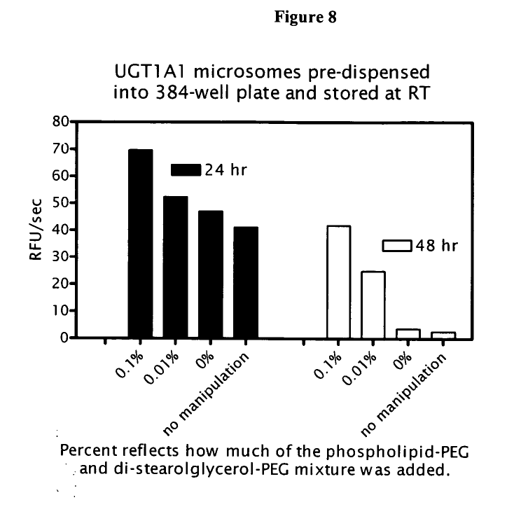

FIG. 8 shows the stability of UGT 1A1 at room temperature for 48 hours. The stability is increased in the presence of the lipid compositions of the present invention.

Binding Affinity of UGT 1A1 Treated with a Mixture of Phospholipid-PEG and Di-Stearolglycerol-PEG

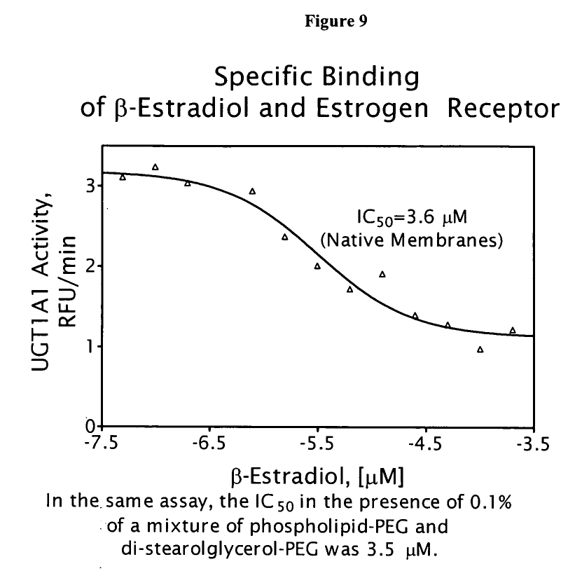

FIG. 9 shows the binding affinity of UGT1A1 treated with lipid compositions of the present invention. Similar IC₅₀ values were obtained for β-estradiol, a specific UGT1A1 substrate, using a membrane preparation stabilized with a mixture of phospholipid-PEG and di-stearolglycerol-PEG and native UGT1A1 membrane preparations.

Assay Tolerance to DMSO

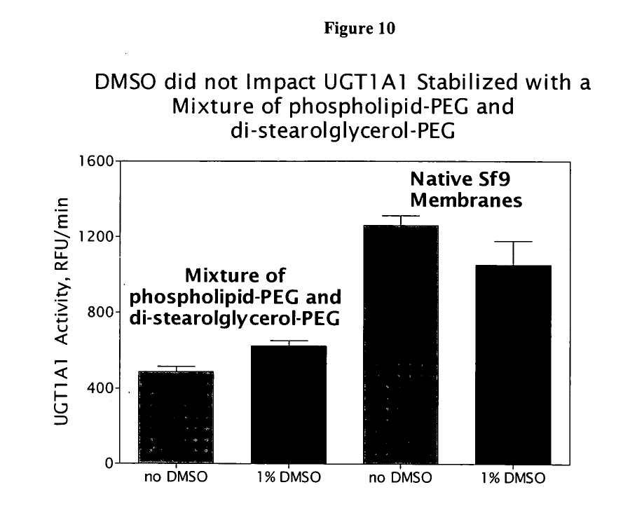

Assay tolerance to DMSO is an important parameter to be determined in HTS applications. FIG. 10 shows that addition of a mixture of phospholipid-PEG and di-stearolglycerol-PEG increases assay tolerance to DMSO.

The Effects of Using a Mixture of Phospholipid-PEG and Di-Stearolglycerol-PEG on Membrane Fractions Used in HTS

352 compounds from the University of Wisconsin high throughpus screening (HTS) library collection were screened using UGT1A1 stabilized with a mixture of phospholipid-PEG and di-stearolglycerol-PEG at 25 μM concentration. Out of all compounds screened, <1.5% exhibited initial fluorescence. About 6% of compounds in this collection were identified as allosteric reporter reaction modifiers (e.g., UGT1A1 substrates or inhibitors). HTS assays utilizing UGT1A1 preparations stabilized with a mixture of phospholipid-PEG and di-stearolglycerol-PEG exhibited a broader dynamic range and resulted in reduced number of assay false positives as evidenced by reduction of the compound's promiscuity.

The Effects of Using a Mixture of Phospholipid-PEG and Di-Stearolglycerol-PEG on Compound Potency

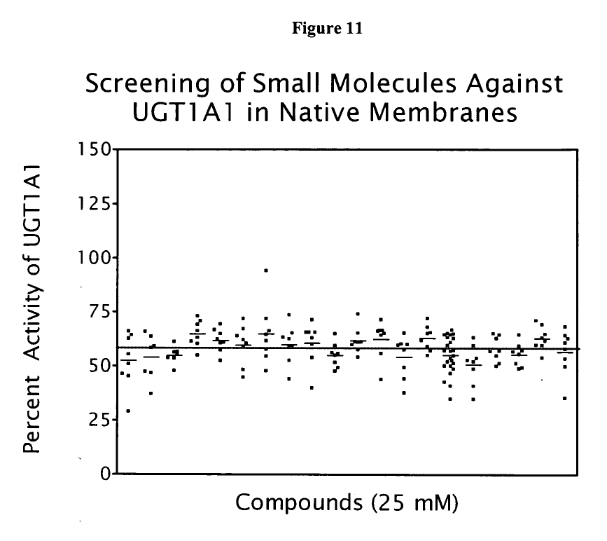

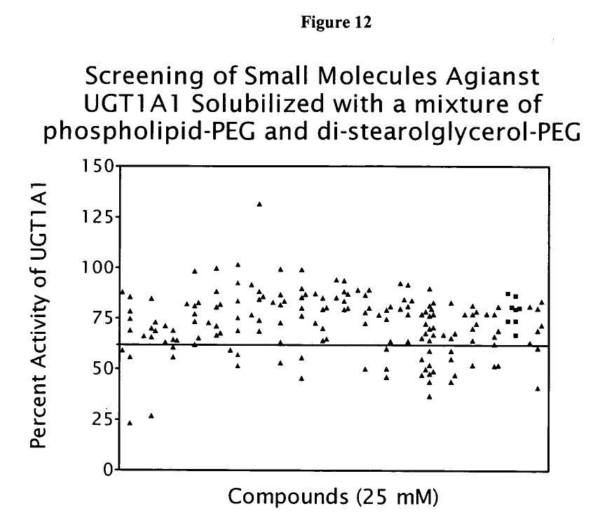

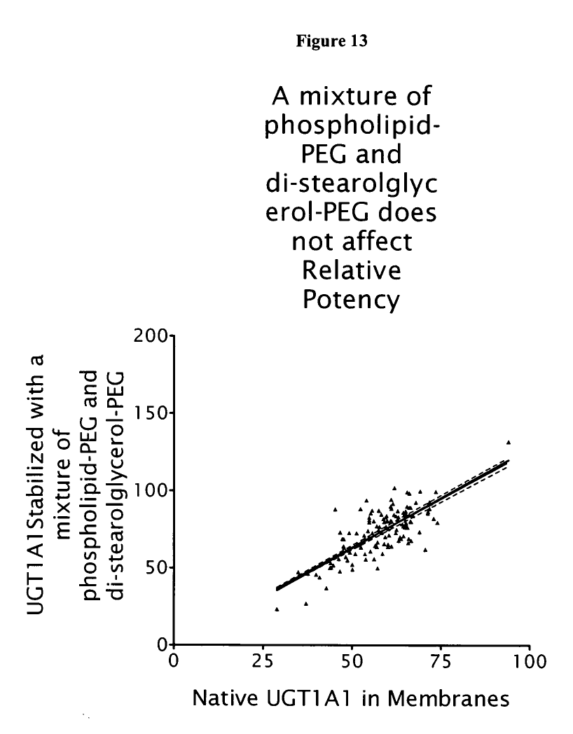

FIGS. 11-13 shows the effect of using a mixture of lipid formulations on compound potency. The results show that the presence of a mixture of phospholipid-PEG and di-stearolglycerol-PEG does not interfere with the ranking order of compound's potency.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising a first negatively charged amphiphilic polymer and a second neutral amphiphilic polymer, and an isolated membrane protein.

2. The composition of claim 1, wherein said first amphiphilic polymer is a phospholipid-polyethylene glycol (PEG) conjugate.

3. The composition of claim 2, wherein said phospholipid comprises a first hydrocarbon chain and a second hydrocarbon chain, and wherein said first and second hydrocarbon chains are between 10 and 20 carbons in length.

4. The composition of claim 3, wherein said first and second hydrocarbon chains are between 14 and 18 carbons in length.

5. The composition of claim 2, wherein said first hydrocarbon chain is a different length than said second hydrocarbon chain.

6. The composition of claim 2, wherein said first and second hydrocarbon chains are saturated.

7. The composition of claim 2, wherein said first and second hydrocarbon chains are partially unsaturated.

8. The composition of claim 1, wherein said second amphiphilic polymer is a diacylglycerol-PEG conjugate.

9. The composition of claim 8, wherein said diacylglycerol comprises a first hydrocarbon chain and a second hydrocarbon chain, and whereins said first and second hydrocarbon chains are between 10 and 20 carbons in length.

10. The composition of claim 9, wherein said first and second hydrocarbon chains are between 14 and 18 carbons in length.

11. The composition of claim 10, wherein said first and second hydrocarbon chains are selected from the group consisting of 14 carbon chains, 16 carbon chains, and 18 carbon chains.

12. The composition of claim 9, wherein said first hydrocarbon chain is a different length than said second hydrocarbon chain.

13. The composition of claim 9, wherein said first and second hydrocarbon chains are saturated.

14. The composition of claim 9, wherein said first and second hydrocarbon chains are partially unsaturated.

15. The composition of claim 1, wherein said composition is configured for the extraction of membrane proteins while maintaining activity of said membrane proteins.

16. The composition of claim 1, wherein at least a portion of at least one of said first and second polymers is covalently attached to a tag.

17. The composition of claim 16, wherein said tag is selected from the group consisting of a label, a biotin, a His tag, GST, an unnatural amino acid, and maltose binding protein.

18. The composition of claim 17, wherein said label is a fluorescent marker.

19. A method, comprising:
a) providing a membrane sample, wherein said membrane sample comprises a membrane component of interest wherein said membrane component is selected from the group consisting of proteins, peptides, carbohydrates, lipids, or combinations thereof; and
b) isolating said membrane component using an amphiphilic polymer complex composition comprising at least one polymer selected from the group consisting of a first negatively charged amphiphilic polymer and a second neutral amphiphilic polymer;
c) exposing said isolated membrane component to a drug; and
d) analyzing the effect of said drug on said isolated membrane component.

20. The method of claim 19, wherein said isolated membrane component is attached to a solid surface prior to said exposing step.
